(19) United States
(12) Patent Application Publication
(10) Pub. No.: US 2015/0352005 A1
(43) Pub. Date: Dec. 10, 2015

(54) Indirect Bird-Pecking Moxibustion Device that is Portable and Automatically Height Adjustable

(71) Applicant: Yuping Sun, Jinan (CN)

(72) Inventor: Yuping Sun, Jinan (CN)

(21) Appl. No.: 14/566,168

(22) Filed: Dec. 10, 2014

(30) Foreign Application Priority Data
Jun. 9, 2014 (CN) 201410249183.3

(51) Int. Cl. A61H 39/06; A61F 7/03

(52) U.S. Cl. CPC A61H 39/06; A61F 7/032

(57) ABSTRACT

An indirect bird-pecking moxibustion device that is portable and automatically height adjustable. The device comprises a hollow tube adapted to hold a moxa cone over an acupuncture point, a locating ring that locates the hollow tube above the acupuncture point, and a mechanism connected to the hollow tube reciprocally moving the hollow tube in relation to the acupuncture point.

The present application hereby claims priority to the Chinese application No. 201410249188.3, filed on Jun. 9, 2014.

### TECHNICAL FIELD

The present invention belongs to the medical device technical field. More specifically, the present invention is an indirect bird-pecking moxibustion device that is portable and automatically height adjustable.

### BACKGROUND

Moxibustion is a traditional Chinese medicine therapy using moxa made from dried mugwort (Artemisia argyi). Although available scientific evidence does not support claims that moxibustion is effective in preventing or treating cancer or any other disease, it plays an important role in the traditional medical systems of China, Japan, Korea, Vietnam, and Mongolia. Suppliers usually age the mugwort and grind it up to a fluff. Practitioners burn the fluff or process it further into a cigar-shaped stick, known as a moxa cone.

There are several methods of moxibustion. Three of them are direct scarring, direct non-scarring, and indirect moxibustion. Direct scarring moxibustion places a small cone of moxa on the skin at an acupuncture point and burns it until the skin blisters, which then scars after it heals. Direct non-scarring moxibustion removes the burning moxa before the skin burns enough to scar, unless the burning moxa is left on the skin too long. Indirect moxibustion requires a practitioner to hold a moxa cone near an acupuncture point to heat the skin, or hold it on an acupuncture needle inserted in the skin to heat the needle. Bird-pecking moxibustion refers to a type of indirect moxibustion that requires a practitioner to hold a burning moxa cone with the burning end over an acupuncture point, and move the cone up and down in a way similar to bird pecking to give the patient a moxibustion with a stronger but intermittent hot stimulation. There is no fixed distance between the moxa cone and the patient to be treated by moxibustion.

When treating patients who suffer from gynecological, digestive system, urinary system, cardiovascular, high blood pressure, endocrine, or orthopedic diseases, or when helping people in enhancing immunity, traditionally it requires a practitioner to hold a lighted moxa cone in his or her hand pointed to related acupuncture points to complete the indirect moxibustion. The practitioner also needs to adjust the distance between the moxa cone and the patient to achieve the most comfortable temperature. These procedures make moxibustion difficult and labor intensive. The treatment has to be one-on-one, which is inefficient. During the process, the patient's activities are limited. Most of the time, he or she has to remain still for a long period of time. Moreover, when multiple patients are treated in a single room, the accumulated smoke will be harmful to the patients. All of those shortcomings mentioned above make the treatment unpleasant.

### SUMMARY

In order to overcome those aforementioned shortcomings, the present invention provides an indirect bird-pecking moxibustion device that is portable and automatically height adjustable. It comprises a hollow tube adapted to hold a moxa cone over an acupuncture point, a locating ring that locates the hollow tube above the acupuncture point, and a mechanism connected to the hollow tube reciprocally moving the hollow tube in relation to the acupuncture point.

In accordance with the invention, patients can complete moxibustion treatments while lying down or standing up. When there is no need to eject smoke generated while burning moxa cone, patients can even be treated while walking. The treatment can be completed either at home, or in a hospital. It will reduce the labor intensity, and also enable practitioners to apply multiple treatments on different patients. The process can also be controlled by computer programs as well.

### DESCRIPTION OF DRAWINGS

FIG. 1 is a perspective schematic view of the invention.

FIG. 2 is an enlarged sectional view of FIG. 1 taken along line A-A in FIG. 1.

FIG. 3 is an enlarged sectional view of FIG. 1 taken along line B-B in FIG. 1.

### DETAILED DESCRIPTION

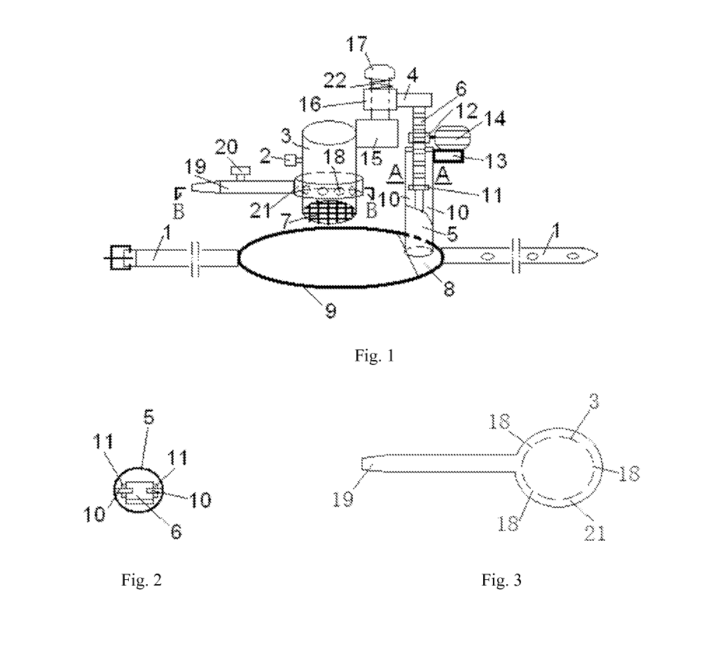

Referring to FIGS. 1, 2 and 3, a moxa cone holding tube 3 connects to the left side of a fixed block 15. The top of the fixed block 15 connects to the bottom of a moving core 17. The moving core 17 inserts into a hollow portion of an electromagnetic bobbin 16. A return spring 22 urges the moving core 17 upward. The electromagnetic bobbin 16 connects to the left side of a horizontal support frame 4. The underside of the horizontal support frame 4 connects to the top of a vertically movable lifting and lowering rack 6. The vertically movable lifting and lowering rack 6 is inserted between two slides 10 located in a tube 5. A limit block 11 is affixed to the bottom of the vertically movable lifting and lowering rack 6. A supporting frame 13 is affixed to the tube 5. A low speed motor 14 that contains gears 12 is affixed to the supporting frame 13. Gears 12 mesh with the vertically movable lifting and lowering rack 6.

The bottom of the tube 5 is affixed to a base 8. The base 8 is affixed to a locating ring 9. Positioning belts 1 are attached to the locating ring 9. Smoke holes 18 are located on the surface of the moxa cone holding tube 3. An evacuation annular tube 21 surrounds the moxa cone holding tube 3 at its lower half, and connects to the moxa cone holding tube 3 through smoke holes 18. The evacuation plug tube 19 equipped with a switch 20 also connects to the evacuation annular tube 21. One or more fastening screws 2 are attached to the moxa cone holding tube 3. An ash receiver 7 is located at the bottom of the moxa cone holding tube 3.

When treating patients with gynecological, digestive system, urinary system, cardiovascular, high blood pressure, endocrine, or orthopedic diseases, or when helping people in enhancing immunity, a practitioner will insert an ignited moxa cone into the moxa cone holding tube 3, with the lighted end of the moxa cone close to the ash receiver 7. After locating the targeted acupuncture point, the practitioner will place the locating ring 9 around the targeted acupuncture point area, and use the positioning belts 1 to fasten and fix the device to a patient's body. If a patient is in need of a stronger treatment, the practitioner can remove the ash receiver 7, and use the fastening screws 2 to directly locate the moxa cone to the targeted acupuncture point area.

The evacuation plug tube 19 is connected to the evacuation annular tube 21, so that when burning the moxa cone to treat patients, smoke will be ejected out of the room through the smoke holes 18, evacuation annular tube 21 and evacuation plug tube 19. By controlling the switch 20, the practitioner will be able to open or close the airway inside of the plug tube 19 to manage smoke ejection. Because the smoke holes 18 are designed to be located close to the bottom of the moxa cone holding tube 3, they will catch all the generated smoke, and ensure oxygen flow to let the moxa cone fully combust and reduce smoke.

When the electromagnetic bobbin 16 is provided with pulsed electricity, the moving core 17 and the return spring 22 will reciprocate up and down to allow the moxa cone to complete a bird-pecking moxibustion on the skin of the patient. The limit block 11 prevents the vertically movable lifting and lowering rack 6 from sliding out of the tube 5.

What is claimed is:

1. A moxibustion device for delivering moxa at an acupuncture point, comprising:
a hollow tube adapted to hold an ignited moxa cone over an acupuncture point, a locating ring that locates the moxa cone above the acupuncture point, and a mechanism connected to the hollow tube reciprocally moving the hollow tube in relation to the acupuncture point.

2. A moxibustion device according to claim 1, wherein the mechanism connected to the hollow tube that reciprocally moves the hollow tube comprises a linkage and a low speed motor that provides vertical placement of the hollow tube.

3. A moxibustion device according to claim 2, wherein the linkage comprises a vertically movable lifting and lowering rack inserted between two slides located in a tube for positioning the moxa cone over the acupuncture point.

4. A moxibustion device according to claim 3, wherein the linkage comprises a limit block fixed to the bottom of the vertically movable lifting and lowering rack to prevent the rack from sliding out of the tube.

5. A moxibustion device according to claim 3, wherein the linkage comprises a horizontal support frame that connects the top of the vertically movable lifting and lowering rack to an electromagnetic bobbin.

6. A moxibustion device according to claim 5, wherein the linkage comprises a moving core that inserts into the electromagnetic bobbin.

7. A moxibustion device according to claim 6, wherein the linkage comprises a return spring in between the moving core and the electromagnetic bobbin, the return spring returning the moving core to an upper position during operation of the bobbin.

8. A moxibustion device according to claim 6, wherein the linkage comprises a fixed block connecting the bottom of the moving core and the hollow tube.

9. A moxibustion device according to claim 1, further comprising an exhaust system to eject smoke.

10. A moxibustion device according to claim 9, wherein the exhaust system connected to the hollow tube comprises smoke holes located on the surface of the hollow tube, an evacuation plug tube equipped with a switch, and an evacuation annular tube that connects to the hollow tube through smoke holes and the evacuation plug tube.

11. A moxibustion device according to claim 1, further comprising one or more fastening screws set upon the hollow tube.

12. A moxibustion device according to claim 1, further comprising an ash receiver set at the bottom of the hollow tube.

13. A moxibustion device according to claim 1, further comprising an external fixation device.

14. A moxibustion device according to claim 13, wherein the external fixation device comprises positioning belts connected to the locating ring.
